(19) United States
(12) Patent Application Publication
(10) Pub. No.: US 2024/0338346 A1
(43) Pub. Date: Oct. 10, 2024

(54) METHODS FOR DATA STORAGE IN MAGNETIC RESONANCE SCANNING, METHODS AND SYSTEMS FOR MAGNETIC RESONANCE SCANNING

(71) Applicant: WUHAN UNITED IMAGING LIFE SCIENCE INSTRUMENT CO., LTD., Wuhan (CN)

(72) Inventors: Rui ZOU, Wuhan (CN); Meiling JI, Wuhan (CN); Renkuan ZHAI, Wuhan (CN)

(73) Assignee: WUHAN UNITED IMAGING LIFE SCIENCE INSTRUMENT CO., LTD., Wuhan (CN)

(21) Appl. No.: 18/744,676

(22) Filed: Jun. 16, 2024

Related U.S. Application Data
(63) Continuation of application No. PCT/CN2022/100375, filed on Jun. 22, 2022.

(30) Foreign Application Priority Data
Dec. 16, 2021 (CN) 202111543322.7

(51) Int. Cl. G06F 16/17; G01R 33/54

(52) U.S. Cl. CPC G06F 16/173; G01R 33/543

(57) ABSTRACT

Disclosed is a method for data storage in magnetic resonance scanning, and a method and system for magnetic resonance scanning, which may include: detecting, during a scanning process, whether a protocol sequence suspending command is triggered; in response to determining that the protocol sequence suspending command is triggered, switching a scanning sequence from a scanning state to a suspending state; when the scanning sequence is in the suspending state, controlling the magnetic resonance device to stop scanning the target object and recording a completed scanning task and a raw data file corresponding to the completed scanning task; detecting whether the user activates the scanning sequence; and in response to determining that the scanning sequence is activated, determining an activation position of the scanning sequence based on a trigger state of the protocol sequence suspending command, and continuing to perform subsequent scanning of the scanning sequence based on the activation position.

### CROSS-REFERENCE TO RELATED APPLICATIONS

This application a Continuation of International Application No. PCT/CN2022/100375, filed on Jun. 22, 2022, which claims priority to Chinese Patent Application No. 202111543322.7, filed on Dec. 16, 2021, the entire contents of each of which are incorporated herein by reference.

### TECHNICAL FIELD

The present disclosure relates to the field of magnetic resonance imaging technology, and in particular, to methods for data storage in magnetic resonance scanning, and methods and systems for magnetic resonance scanning.

### BACKGROUND

Magnetic resonance imaging is an imaging technique that reconstructs an image from a signal generated by resonance of atomic nuclei in a strong magnetic field. With the development of medical technology, the use of magnetic resonance imaging technology in medical diagnostics has become increasingly common.

When scanning humans or animals using magnetic resonance imaging technology, especially when employing an ultra-high-field magnetic resonance device (e.g., a 9.4 T magnetic resonance device) for life science research on animals, some experiments require a long scanning time, for example, continuous scanning for dozens of hours or even days, resulting in excessively large raw data files generated from the scan. If a generated raw image is too large, relevant original medical image data may not be obtained quickly and accurately for subsequent image reconstruction. On the other hand, during a scanning process, an unexpected factor may cause the scanning process to be terminated, resulting in a failure of the scanning process, and raw data obtained in an early stage of the scanning process has to be discarded, requiring the scanning process to be restarted.

Therefore, it is desirable to provide a method for data storage in magnetic resonance scanning, and a method and a system for magnetic resonance scanning to manage the scanning process and scanning data, thereby improving scanning efficiency.

### SUMMARY

One embodiment of the present disclosure provides a method for data storage in magnetic resonance scanning. The method may be implemented on at least one machine each of which may have at least one processor and a storage device, and the method may include: obtaining a raw data file of a current scanning layer during scanning a target object using a magnetic resonance device, determining a target file group corresponding to the raw data file of the current scanning layer according to a preset data slicing rule, and storing data of the raw data file of the current scanning layer into a storage space corresponding to the target file group.

One embodiment of the present disclosure provides a system for magnetic resonance scanning, which may include a protocol manager module, a checklist module, and a data storage module. The protocol manager module may be configured to obtain a target scanning protocol selected by a user via a protocol scanning interface and transmit the target scanning protocol to the checklist module. The checklist module may be configured to navigate to a slice selection interface based on the target scanning protocol, obtain a preset data slicing rule selected by the user via the slice selection interface, and scan a target object to generate a raw data file of a current scanning layer. The data storage module may be configured to store the raw data file of the current scanning layer according to the preset data slicing rule.

One embodiment of the present disclosure provides a method for magnetic resonance scanning executed by a magnetic resonance device. The method may be implemented on at least one machine each of which may have at least one processor and a storage device, and the method may include: obtaining a target scanning protocol selected by a user via a protocol scanning interface; navigating to a slice selection interface based on the target scanning protocol, obtaining a preset data slicing rule selected by the user via the slice selection interface, and scanning a target object to generate a raw data file of a current scanning layer; and storing the raw data file of the current scanning layer according to the preset data slicing rule, and storing data of the raw data file of the current scanning layer into a storage space corresponding to a target file group.

One embodiment of the present disclosure provides a system for magnetic resonance scanning, which may include a suspending module and an activation module. The suspending module may be configured to obtain a protocol sequence suspending command based on the protocol scanning interface and when the protocol sequence suspending command is received, control a magnetic resonance device to stop scanning the target object, record a layer count of scanning layers that have been scanned, generate a task execution log file, and transmit the task execution log file to the data storage module for storage. The activation module may be configured to obtain a suspending protocol sequence activation command based on the protocol scanning interface, search for the task execution log file in the data storage module, retrieve the layer count of scanning layers that have been scanned, and scan layers that have not been scanned.

One embodiment of the present disclosure provides a method for magnetic resonance scanning executed by a magnetic resonance device. The method may be implemented on at least one machine each of which may have at least one processor and a storage device, and the method may include: detecting, during a scanning process, whether a protocol sequence suspending command is triggered; in response to determining that the protocol sequence suspending command is triggered, switching a scanning sequence from a scanning state to a suspending state; when the scanning sequence is in the suspending state, controlling the magnetic resonance device to stop scanning the target object and recording a completed scanning task and a raw data file corresponding to the completed scanning task, wherein the scanning task may be associated with at least one of scanned layers of the magnetic resonance scanning and a magnetic resonance signal corresponding to each of the scanned layers; detecting whether the user activates the scanning sequence; and in response to determining that the user activates the scanning sequence, determining an activation position of the scanning sequence based on a trigger state of the protocol sequence suspending command, and continuing to perform subsequent scanning of the scanning sequence based on the activation position.

One embodiment of the present disclosure provides a system for magnetic resonance scanning, which may include a suspending module and an activation module. The suspending module detect may be configured to: detect, during a scanning process, whether a protocol sequence suspending command is triggered; in response to determining that the protocol sequence suspending command is triggered, switch a scanning sequence from a scanning state to a suspending state; when the scanning sequence is in the suspending state, control the magnetic resonance device to stop scanning the target object and record a completed scanning task and a raw data file corresponding to the completed scanning task, wherein the scanning task may be associated with at least one of scanned layers of the magnetic resonance scanning, and a magnetic resonance signal corresponding to each of the scanned layers. The activation module may be configured to: detect whether the user activates the scanning sequence; and in response determining that the user activates the scanning sequence, determine an activation position of the scanning sequence based on a trigger state of the protocol sequence suspending command, and continue to perform subsequent scanning of the scanning sequence based on the activation position.

One embodiment of the present disclosure provides a device for magnetic resonance scanning. The device may include at least one storage medium storing one or more computer commands and at least one processor executing the one or more computer commands to implement the method for data storage in magnetic resonance scanning and the method for magnetic resonance scanning.

One embodiment of the present disclosure provides a non-transitory computer-readable storage medium. The storage medium may store one or more computer commands, and when a computer reads the one or more computer commands from the storage medium, the computer executes the method for data storage in magnetic resonance scanning and the method for magnetic resonance scanning.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram of an exemplary application scenario of a system for magnetic resonance scanning according to some embodiments of the present disclosure;

FIG. 2 is a schematic diagram of exemplary modules of a system for magnetic resonance scanning according to some embodiments of the present disclosure;

FIG. 3 is a flowchart of an exemplary process for magnetic resonance scanning according to some embodiments of the present disclosure;

FIG. 4 is a flowchart of an exemplary process for determining an activation position according to some embodiments of the present disclosure;

FIG. 5 is a schematic diagram of an exemplary application scenario of magnetic resonance scanning according to some embodiments of the present disclosure;

FIG. 6A is a flowchart of an exemplary process for data storage in magnetic resonance scanning according to some embodiments of the present disclosure;

FIG. 6B is a schematic diagram of an exemplary raw data slice selection interface according to some embodiments of the present disclosure;

FIG. 7 is a flowchart of an exemplary process for data storage in magnetic resonance scanning according to some embodiments of the present disclosure;

FIG. 8 is a flowchart of an exemplary process for data storage in magnetic resonance scanning according to some embodiments of the present disclosure;

FIG. 9A is a flowchart of an exemplary process for data storage in magnetic resonance scanning according to some embodiments of the present disclosure;

FIG. 9B is a schematic diagram of an exemplary protocol scanning interface according to some embodiments of the present disclosure;

FIG. 9C is a schematic diagram of an exemplary scanning sequence resume interface according to some embodiments of the present disclosure;

FIG. 9D is a schematic diagram of an exemplary checklist interface according to some embodiments of the present disclosure;

FIG. 10 is a flowchart of an exemplary process for data storage in magnetic resonance scanning according to some embodiments of the present disclosure;

FIG. 11 is a structural diagram of an exemplary system for magnetic resonance scanning according to some embodiments of the present disclosure;

FIG. 12 is a flowchart of an exemplary process for magnetic resonance scanning according to some embodiments of the present disclosure; and

FIG. 13 is a schematic diagram of an internal structural of an exemplary electronic device according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Flowcharts are used in the present disclosure to illustrate the operations performed by the system according to some embodiments of the present disclosure. It should be understood that the operations described herein are not necessarily executed in a specific order. Instead, the operations may be executed in reverse order or simultaneously. Additionally, one or more other operations may be added to these processes, or one or more operations may be removed from these processes.

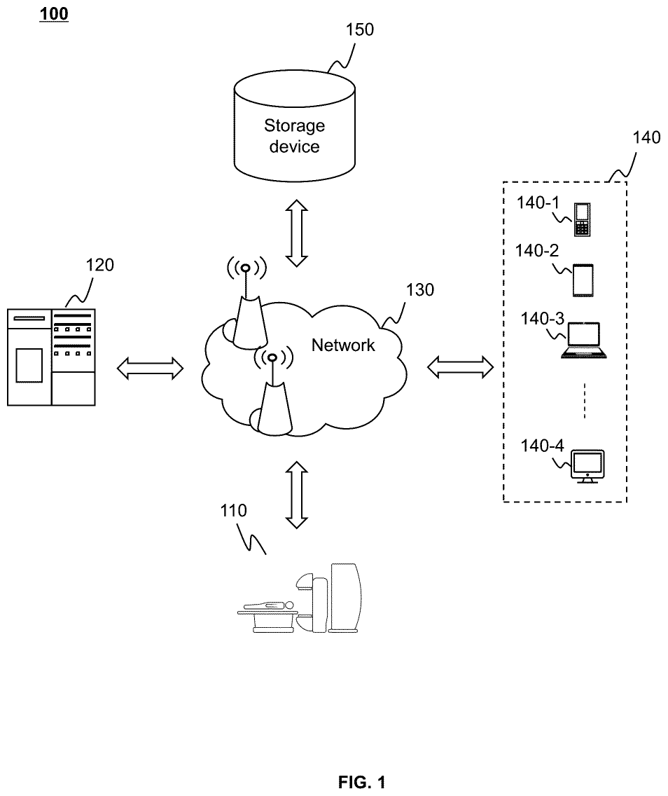

FIG. 1 is a schematic diagram of an exemplary application scenario of a system for magnetic resonance scanning according to some embodiments of the present disclosure.

In some embodiments, as shown in FIG. 1, a system 100 for magnetic resonance scanning may include a magnetic resonance device 110, a processing device 120, a network 130, at least one terminal device 140, and a storage device 150. In some embodiments, various components of the system 100 for magnetic resonance scanning may be interconnected via the network 130. In some embodiments, a portion of the components of the system 100 for magnetic resonance scanning may be directly connected.

The magnetic resonance device 110 may be used to perform magnetic resonance scanning on a target object. In some embodiments, the target object may be an animal, such as an experimental mouse. When conducting preclinical life science research by performing magnetic resonance scanning on the animal, the corresponding magnetic resonance device 110 may be an ultra-high-field magnetic resonance device (e.g., a 7 T magnetic resonance device, a 9.4 T magnetic resonance device, or an 11 T magnetic resonance device). In some embodiments, the target object may also include other organisms or non-living objects besides the animal. For example, the target object may include a patient, an artificial object, etc. In some embodiments, the target object may include a specific part of a human body, such as the head, neck, chest, or any combination thereof. In some embodiments, the target object may include a specific organ, such as the liver, kidneys, pancreas, bladder, uterus, rectum, or any combination thereof. In some embodiments, the target object may include a region of interest (ROI), such as a tumor, a nodule, etc. In some embodiments, the magnetic resonance device 110 may obtain a large amount of unprocessed primary data (also referred to as raw data) by performing the magnetic resonance scanning on the target object. In some embodiments, the magnetic resonance device 110 may perform the magnetic resonance scanning on the target object according to a scanning sequence, where the scanning sequence may include but is not limited to a free induction decay (FID) sequence, a spin echo (SE) sequence, a gradient echo (GRE) sequence, a hybrid sequence (HS), etc. In some embodiments, different scanning sequences may be used for scanning different target objects. For example, an Ax SE T1 scanning sequence may be used for a routine head scan, and a Cor SE T1 scanning sequence may be used for a pituitary scan.

The above description of the magnetic resonance device 110 is for illustrative purposes only and is not intended to limit the scope of the present disclosure.

In some embodiments, the magnetic resonance device 110 may transmit scanning data to the processing device 120 and/or other components of the system 100 for magnetic resonance scanning. In some embodiments, the magnetic resonance device 110 may receive relevant data or commands from the processing device 120 and/or other components of the system 100 for magnetic resonance scanning to perform the magnetic resonance scanning.

The processing device 120 may process data and/or information obtained from the magnetic resonance device 110, the at least one terminal device 140, and/or the storage device 150. For example, the processing device 120 may obtain a target scanning protocol selected by a user based on a protocol scanning interface, retrieve a preset slicing rule selected by the user based on a slice selection interface, and control the magnetic resonance device 110 to scan the target object, thereby generating a raw data file of a current scanning layer. The processing device 120 may transmit the raw data file of the current scanning layer to a corresponding storage space in the storage device 150 based on the preset data slicing rule for storage. As another example, during an execution process of the magnetic resonance device 110, the processing device 120 may detect whether a protocol sequence suspending command is triggered, and in response to determining that the protocol sequence suspending command is triggered, switch the scanning sequence from a scanning state to a suspending state, control the magnetic resonance device to stop scanning the target object, and record a completed scanning task and a raw data file corresponding to the completed scanning task. The processing device 120 may detect whether the user activates the scanning sequence, and in response to determining that the user activates the scanning sequence, determine an activation position of the scanning sequence based on a trigger state of the protocol sequence suspending command, and continue to perform subsequent scanning of the scanning sequence based on the activation position.

In some embodiments, the processing device 120 may be a single server or a server group. The server group may be centralized or distributed. In some embodiments, the processing device 120 may be local or remote. For example, the processing device 120 may access information and/or data from the magnetic resonance device 110, the at least one terminal device 140, and/or the storage device 150 via the network 130. As another example, the processing device 120 may be directly connected to the magnetic resonance device 110, the at least one terminal device 140, and/or the storage device 150 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or any combination thereof.

The network 130 may include any suitable network capable of facilitating the exchange of information and/or data for the system 100 for magnetic resonance scanning. In some embodiments, one or more components (e.g., the magnetic resonance device 110, processing device 120, the at least one terminal device 140, the storage device 150, etc.) of the system 100 for magnetic resonance scanning may exchange information via the network 130. For example, the processing device 120 may receive scan operation stream data (i.e., operation stream data during scanning executed by the magnetic resonance device 110) from the magnetic resonance device 110 via the network 130. As another example, the processing device 120 may read data stored in the storage device 150 via the network 130.

The at least one terminal device 140 may communicate and/or connect with the magnetic resonance device 110, the processing device 120, and/or the storage device 150. For example, the at least one terminal device 140 may send one or more control commands to the magnetic resonance device 110 via the network 130 to control the magnetic resonance device 110 to perform magnetic resonance scanning on the target object according to the one or more control commands. In some embodiments, the at least one terminal device 140 may include one of a mobile device 140-1, a tablet 140-2, a laptop 140-3, a desktop computer 140-4, or other devices with input and/or output functions, or a combination thereof. In some embodiments, the at least one terminal device 140 may include an input device, an output device, etc. The input device may include a keyboard, a touchscreen, a mouse, a voice device, or the like, or any combination thereof. The output device may include a display, a speaker, a printer, or the like, or any combination thereof. In some embodiments, the at least one terminal device 140 may be a part of the processing device 120. In some embodiments, the at least one terminal device 140 may be integrated with the processing device 120 as an operating console of the magnetic resonance device 110.

The storage device 150 may store data, commands, and/or any other information. In some embodiments, the storage device 150 may store a raw data file obtained by the magnetic resonance device 110. In some embodiments, the storage device 150 may store data obtained from the magnetic resonance device 110, the processing device 120, and/or the at least one terminal device 140. For example, when the scanning sequence in the magnetic resonance device 110 is in a suspending state, the processing device 120 may control the magnetic resonance device 110 to stop scanning the target object, record the completed scanning task and the raw data file corresponding to the completed scanning task, and store a task execution log file to the storage device 150. As another example, the processing device 120 may store a complete raw data file in the storage device 150 when the magnetic resonance device 110 completes scanning the target object. In some embodiments, the storage device 150 may store data and/or commands used by the processing device 120 to execute or implement the exemplary processes described in the present disclosure.

In some embodiments, the storage device 150 may include a large capacity storage, a removable storage, a volatile read-write memory, a read-only memory (ROM), or the like, or any combination thereof. For example, the large capacity storage includes a disk, an optical disk, a solid-state disk, etc. For example, the removable storage includes a flash drive, a floppy disk, an optical disk, a memory card, a zip disk, a magnetic tape, etc. For example, the volatile read-write memory includes a random access memory (RAM), etc. For example, the RAM includes a dynamic random access memory (DRAM), a double data rate synchronous dynamic random access memory (DDRSDRAM), a static random access memory (SRAM), a thyristor random access memory (T-RAM), a zero-capacitor random access memory (Z-RAM), etc. For example, the ROM includes a mask ROM (MROM), a programmable ROM (PROM), an erasable programmable ROM (EPROM), an electrically erasable programmable ROM (EEPROM), a CD-ROM, a digital versatile disc read-only memory (DVD-ROM), etc. In some embodiments, the storage device 150 may be implemented on a cloud platform.

In some embodiments, the storage device 150 may be connected to the network 130 to communicate with at least one other component (e.g., the magnetic resonance device 110, the processing device 120, the at least one terminal device 140) in the system 100 for magnetic resonance scanning. The at least one component in the system 100 for magnetic resonance scanning may access data or commands stored in the storage device 150 via the network 130. In some embodiments, the storage device 150 may be a part of the processing device 120.

It should be noted that the above descriptions are provided for illustrative purposes only and are not intended to limit the scope of the present disclosure. For those skilled in the art, various changes and modifications may be made under the guidance of the content of the present disclosure. The features, structures, methods, and other features of the exemplary embodiments described in the present disclosure may be combined in various ways to obtain additional and/or alternative exemplary embodiments. For example, the magnetic resonance device 110, the processing device 120, and the at least one terminal device 140 may share a storage device 150, or they may each have their own storage device. However, these changes and modifications do not depart from the scope of the present disclosure.

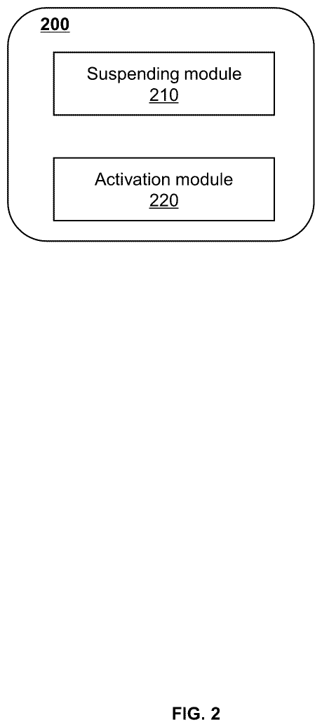

FIG. 2 is a schematic diagram of exemplary modules of a system for magnetic resonance scanning according to some embodiments of the present disclosure.

In some embodiments, as shown in FIG. 2, a system 200 for magnetic resonance scanning may include a suspending module 210 and an activation module 220. In some embodiments, the system 200 for magnetic resonance scanning may be implemented via the processing device 120.

The suspending module 210 may be configured to: detect, during a scanning process, whether a protocol sequence suspending command is triggered; in response to determining that the protocol sequence suspending command is triggered, switch a scanning sequence from a scanning state to a suspending state; when the scanning sequence is in the suspending state, control a magnetic resonance device to stop scanning a target object and record a completed scanning task and a raw data file corresponding to the completed scanning task, wherein the scanning task may be associated with at least one of scanned layers of the magnetic resonance scanning, and a magnetic resonance signal corresponding to each of the scanned layers.

In some embodiments, the suspending module 210 may be further configured to record the completed scanning task and the raw data file corresponding to the completed scanning task in a task execution log file.

The activation module 220 may be configured to: detect whether a user activates the scanning sequence; and in response determining that the user activates the scanning sequence, determine an activation position of the scanning sequence based on a trigger state of the protocol sequence suspending command, and continue to perform subsequent scanning of the scanning sequence based on the activation position.

In some embodiments, the activation module 220 may be further configured to: in response to determining that the trigger state is a first trigger state, determine a first suspension position of a first scanning task, wherein the first scanning task may be a currently executing scanning task recorded when the scanning sequence is suspended; and determine the first suspension position as the activation position. In some embodiments, the activation module 220 may be further configured to: in response to determining that the trigger state is a second trigger state, determine a second suspension position of a second scanning task, wherein the second scanning task may be a preceding completed scanning task recorded when the scanning sequence is suspended; and determine the second suspension position as the activation position.

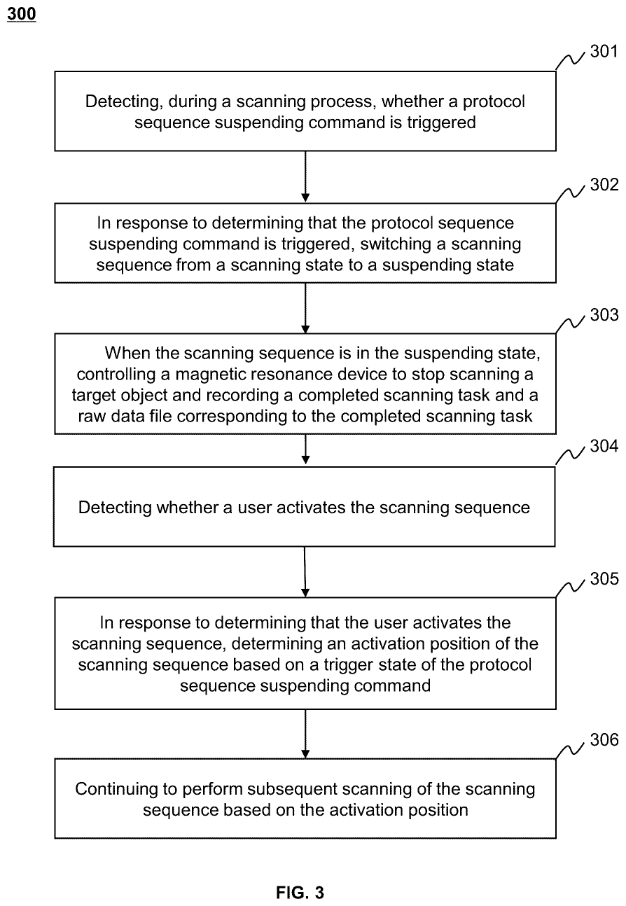

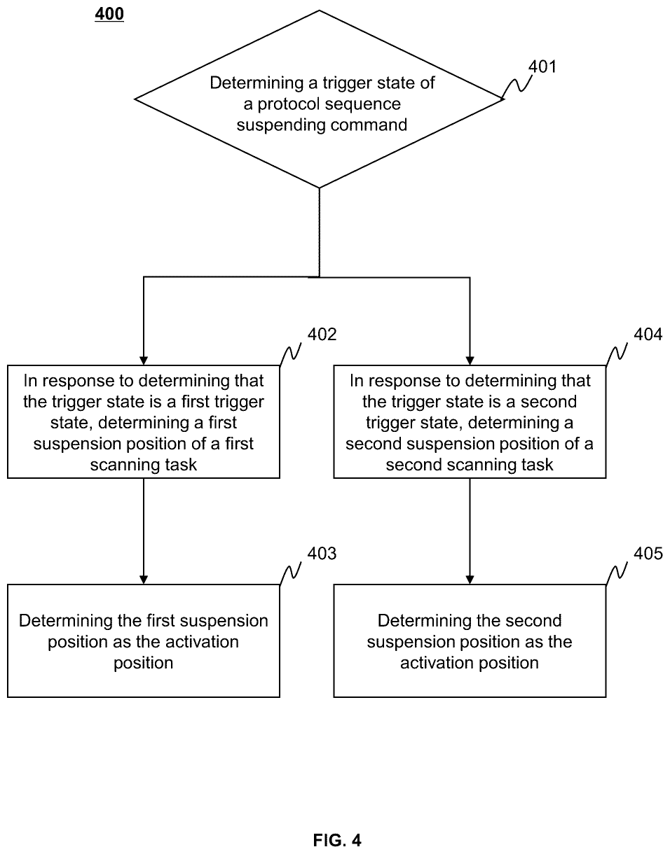

More descriptions regarding the suspending module 210 and the activation module 220 may be found in FIGS. 3-4 and the related descriptions thereof.

It should be noted that the descriptions of the system 200 for magnetic resonance scanning and the modules above is for convenience of description and are not intended to limit the present disclosure to the exemplary embodiments provided. It may be understood that for those skilled in the art, after understanding the principles of the system, various modules may be arbitrarily combined without deviating from these principles, or subsystems may be formed to connect with other modules. In some embodiments, the suspending module 210 and the activation module 220 may be different modules in the same system, or one module may implement the functions of the above two or more modules. For example, each module may share a storage module, or each module may have its own storage module. Such variations are within the scope of the present disclosure.

FIG. 3 is a flowchart of an exemplary process for magnetic resonance scanning according to some embodiments of the present disclosure. In some embodiments, magnetic resonance scanning may be performed by one or more components (e.g., the processing device 120) of the system 100 for magnetic resonance scanning or the system 200 for magnetic resonance scanning. For example, process 300 may be stored in a storage device (e.g., the storage device 150) in the form of a program or commands, and when executed by the processing device 120 or the system 200 for magnetic resonance scanning, the process 300 may be implemented. The descriptions of operations of process 300 provided below is illustrative. In some embodiments, one or more additional operations not described and/or one or more operations not discussed may be used to complete the process 300. In addition, the sequence of the operations of process 300 shown in FIG. 3 and described below is not intended to be limiting.

In 301, whether a protocol sequence suspending command is triggered during a scanning process may be detected. In some embodiments, operation 301 may be performed by the suspending module 210.

The protocol sequence suspending command may be used to suspend a scanning sequence that is currently being executed. Suspending the scanning sequence refers to stopping currently executing scanning. In some embodiments, the protocol sequence suspending command may be a control command issued by a processing device (e.g., the processing device 120) to suspend the scanning sequence that is currently being executed. In some embodiments, the protocol sequence suspending command may be pre-stored in a storage device (e.g., the storage device 150), and when the protocol sequence suspending command is triggered, the storage device may send the protocol sequence suspending command to the processing device to perform subsequent operations.

In some embodiments, when an abnormality occurs during a scanning process, or a user actively triggers the protocol sequence suspending command, the protocol sequence suspending command may be triggered. Accordingly, the suspending module 210 may detect whether the protocol sequence suspending command is triggered by detecting if an abnormality occurs during the scanning process and whether the user actively triggers the protocol sequence suspending command. For example, when detecting an abnormality (e.g., a scanning failure or a crash) during the scanning process, the suspending module 210 may determine that the protocol sequence suspending command is triggered. As another example, when detecting that the user actively triggers the protocol sequence suspending command (e.g., when detecting that the user clicks a “suspend” button on an operation interface to actively trigger the protocol sequence suspending command), the suspending module 210 may determine that the protocol sequence suspending command is triggered.

In 302, in response to determining that the protocol sequence suspending command is triggered, a scanning sequence may be switched from a scanning state to a suspending state. In some embodiments, operation 302 may be performed by the suspending module 210.

The scanning state refers to a state indicating that the scanning sequence is performing scanning. The suspending state refers to a state indicating that the scanning sequence is suspended and the scanning is stopped. In some embodiments, the suspending state may include a state of abnormal suspension and a state of active suspension. The abnormal suspension refers to a suspension of the scanning sequence due to an abnormality occurring during the scanning process. For example, the abnormal suspension may include a suspension of the scanning sequence due to the scanning failure or the crash during the scanning process. The active suspension refers to a suspension of the scanning sequence caused by the user actively triggering the protocol sequence suspending command. For example, the active suspension may include a suspension of the scanning sequence caused by the user actively triggering the protocol sequence suspending command through an option or a button on a terminal device (e.g., the at least one terminal device 140).

In some embodiments, after the protocol sequence suspending command is triggered, the scanning sequence may be in the state of abnormal suspension or active suspension. Accordingly, the suspending module 210 may switch the scanning sequence from the scanning state to the suspending state. For example, when an abnormality occurs during the scanning process and the protocol sequence suspending command is triggered, the scanning sequence is in the state of abnormal suspension, and the suspending module 210 may switch the scanning sequence from the scanning state to the suspending state accordingly. As another example, when the user actively triggers the protocol sequence suspending command, the scanning sequence is in the state of active suspension, and the suspending module 210 may switch the scanning sequence from the scanning state to the suspending state accordingly.

In 303, when the scanning sequence is in the suspending state, a magnetic resonance device may be controlled to stop scanning a target object, and a completed scanning task and a raw data file corresponding to the completed scanning task may be recorded. In some embodiments, operation 303 may be performed by the suspending module 210.

The scanning task refers to a task executed by the magnetic resonance device during scanning. For example, the scanning task may be scanning a certain layer of the target object. In some embodiments, the scanning task may be associated with at least one of scanned layers of the magnetic resonance scanning and a magnetic resonance signal corresponding to each of the scanned layers. A scanning layer may be a three-dimensional structure with a certain layer thickness obtained by filling and superimposing a plurality of pieces of line data obtained from scanning in a K-space. The magnetic resonance signal corresponding to each of the scanned layers may be a magnetic resonance signal corresponding to each position within the scanned layer.

In some embodiments, when performing the magnetic resonance scanning on the target object, a plurality of scanning tasks may be predefined, and each scanning task may correspond to its own label. A plurality of labels corresponding to the plurality of scanning tasks may also indicate a scanning order of the plurality of scanning tasks. For example, when four scanning tasks are predefined and labeled with Scanning Task 1, Scanning Task 2, Scanning Task 3, and Scanning Task 4, respectively, a scanning order of the four scanning tasks may be sequentially Scanning Task 1, Scanning Task 2, Scanning Task 3, and Scanning Task 4. In some embodiments, when a scanning task is completed, the label corresponding to the scanning task may be recorded, and then whether the scanning task is completed may be determined based on whether the label of the scanning task is recorded. For example, if Scanning Task 1 and Scanning Task 3 are recorded, it may be determined that Scanning Task 1 and Scanning Task 3 are completed, while Scanning Task 2 and Scanning Task 4 are not completed.

In some embodiments, different scanning sequences may be used for different scanning tasks, and the scanning tasks and scanning sequences may be predefined and stored in the storage device. For example, Scanning Task 1 may correspond to an AX FSE T1WI sequence, Scanning Task 2 may correspond to a SAG FSE T1WI sequence, Scanning Task 3 may correspond to a COR FSE T1WI sequence, and Scanning Task 4 may correspond to an AX 3D SPGR T1WI sequence.

The raw data file refers to a file formed by a large amount of unprocessed raw data obtained after performing the magnetic resonance scanning on the target object. In some embodiments, after a scanning task is completed, a raw data file corresponding to the scanning task may be generated.

In some embodiments, the suspending module 210 may control the magnetic resonance device to stop scanning the target object when the scanning sequence is in the suspending state. For example, the suspending module 210 control the magnetic resonance device to stop scanning the target object when the scanning sequence is suspended abnormally (i.e., the abnormal suspension) or actively (i.e., the active suspension).

In some embodiments, when the magnetic resonance device stops scanning the target object, the suspending module 210 may record the completed scanning task and the raw data file corresponding to the completed scanning task. For example, for Scanning Task 1, Scanning Task 2, Scanning Task 3, and Scanning Task 4, when the magnetic resonance device stops scanning the target object after Scanning Task 1 and Scanning Task 2 are completed, while Scanning Task 3 is executed and Scanning Task 4 has not started, the suspending module 210 may record the completed Scanning Task 1 and the completed Scanning Task 2 and raw data files a and b corresponding to the completed Scanning Task 1 and the completed Scanning Task 2. In some embodiments, the suspending module 210 may record the label of the completed scanning task and bind the label with the completed scanning task and the raw data file corresponding to the completed scanning task for storage.

In some embodiments, the completed scanning task and the raw data file corresponding to the completed scanning task may be recorded in a task execution log file. The task execution log file may be used to record an execution situation of the scanning task. In some embodiments, the task execution log file may include the label of the completed scanning task, a scanning time (i.e., a scanning duration spent by the scanning task), a completion time (i.e., a time point when the scanning task is completed), and the raw data file corresponding to the completed scanning task. For example, the task execution log file may include a label “1” of the completed Scanning Task 1, a scanning time of 20 minutes, a completion time of 17:30, and a corresponding raw data file “a”.

In some embodiments, when the magnetic resonance device stops scanning the target object, the suspending module 210 may also record a scanning situation of an incomplete scanning task. The scanning situation may include an interruption position and a scanning state of the incomplete scanning task. The interruption position refers to an end position of a completed part of the incomplete scanning task, and the scanning state of the incomplete scanning task may be “incomplete.” For example, when the magnetic resonance device stops scanning the target object, Scanning Task 1 and Scanning Task 2 are completed and Scanning Task 3 is being executed, the suspending module 210 may record Scanning Task 1, Scanning Task 2, and the raw data files corresponding to Scanning Task 1 and Scanning Task 2. At the same time, the suspending module 210 may record that the scanning situation of Scanning Task 3 is that the interruption position is a “position point a” and the scanning state is “incomplete.”

In some embodiments, the raw data file corresponding to the completed scanning task may be stored in a storage space. In some embodiments, a plurality of raw data files may be split and stored the in the storage space according to a data slicing rule.

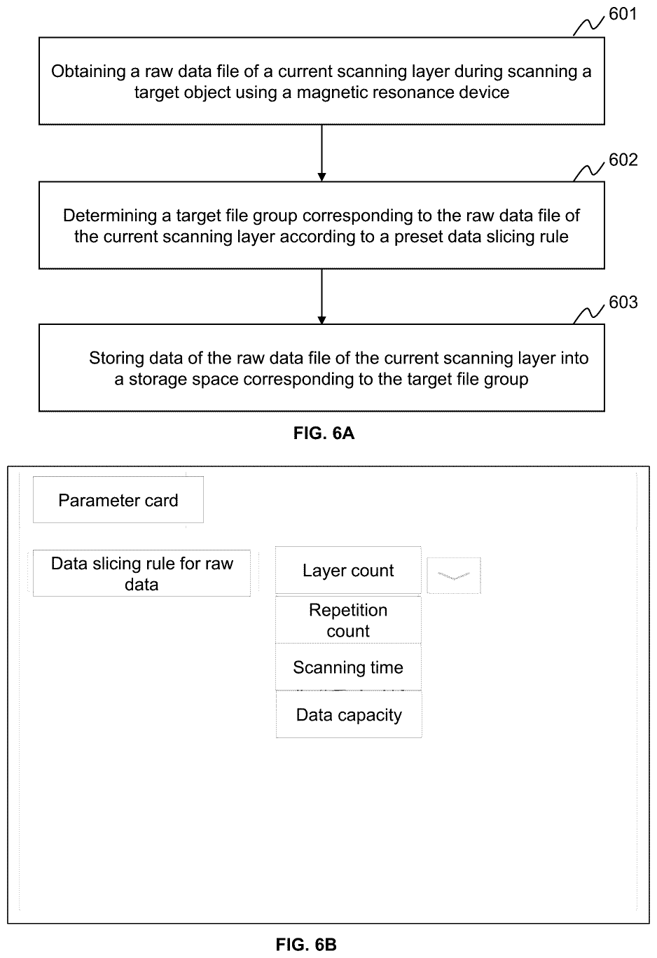

The data slicing rule refers to a rule used for splitting data files before storage. In some embodiments, the data slicing rule may be configured by the user. The data slicing rule may include at least one of: a first rule for grouping raw data files based on a layer count, a second rule for grouping raw data files based on a scanning time, or a third rule for grouping raw data files based on a data capacity. More descriptions of the first rule, the second rule, and the third rule may be found in FIGS. 6A-8 and FIG. 10 and the related descriptions thereof.

In 304, whether the user activates the scanning sequence may be detected. In some embodiments, operation 304 may be executed by the activation module 220.

In some embodiments, after the magnetic resonance device stops scanning, the activation module 220 may detect whether the user activates the scanning sequence. The operation of activating the scanning sequence refers to an operation of restoring the scanning sequence from the suspending state to the scanning state.

In some embodiments, the user may activate the scanning sequence from the suspending state to the scanning state using an activation option or an activation button on the terminal device. Accordingly, when the activation module 220 detects that the activation option or the activation button is triggered, it means that the user activates the scanning sequence. For example, when the user selects the activation option or presses the activation button on the terminal device, it indicates that the user activates the scanning sequence.

In 305, in response determining that the user activates the scanning sequence, an activation position of the scanning sequence may be determined based on a trigger state of the protocol sequence suspending command. In some embodiments, operation 305 may be executed by the activation module 220.

The trigger state refers to a manner in which the protocol sequence suspending command is triggered. For example, the trigger state may include that the protocol sequence suspending command is triggered due to an abnormality occurring during the scanning process, and the user actively triggers the protocol sequence suspending command.

In some embodiments, the trigger state may include a first trigger state and a second trigger state. The first trigger state refers to the user actively triggering the protocol sequence suspending command, and the second trigger state refers to the protocol sequence suspending command being triggered due to an abnormality occurring during the scanning process.

The activation position refers to a position where the scanning process restarts after the scanning sequence is activated. For example, the activation position may be Scanning Task 2, that is, the scanning process may restart scanning from Scanning Task 2.

In some embodiments, the activation module 220 may determine the activation position of the scanning sequence based on the recorded raw data file(s) and the scanning order of scanning tasks. In some embodiments, the activation module 220 may determine a next scanning task after a scanning task corresponding to a last recorded raw data file as the activation position based on the label(s) of the recorded raw data file(s) and the scanning order of the scanning tasks. For example, if the scanning order of the scanning tasks is Scanning Task 1, Scanning Task 2, Scanning Task 3, and Scanning Task 4 sequentially, and the last recorded raw data file corresponds to Scanning Task 3, then the activation module 220 may determine Scanning Task 4 as the activation position.

In some embodiments, the activation module 220 may determine the activation position of the scanning sequence based on the trigger state.

In some embodiments, in response to determining that the trigger state is the first trigger state, the activation module 220 may determine a first suspension position of a first scanning task. The first scanning task refers to a currently executing scanning task recorded when the scanning sequence is suspended. Furthermore, the activation module 220 may determine the first suspension position as the activation position.

In some embodiments, in response to determining that the trigger state is the second trigger state, the activation module 220 may determine a second suspension position of a second scanning task. The second scanning task refers to a preceding completed scanning task recorded when the scanning sequence is suspended.

Furthermore, the activation module 220 may determine the second suspension position as the activation position.

More descriptions of determining the activation position of the scanning sequence may be found in FIG. 4 and the related descriptions thereof.

In 306, subsequent scanning of the scanning sequence may be continued based on the activation position. In some embodiments, operation 306 may be executed by the activation module 220.

In some embodiments, after determining the activation position, the activation module 220 may designate the activation position as a starting point for resuming scanning and continue to perform the subsequent scanning of the scanning sequence from the activation position. For example, if Scanning Task 4 is determined as the activation position, the activation module 220 may continue to perform the subsequent scanning from Scanning Task 4.

In some embodiments of the present disclosure, the suspension and activation of the scanning sequence are controlled when scanning fails, the scanning process crashes, or the user requests to pause the scan, which effectively avoids the risk of invalidating data of previous scanning due to an interruption, thereby improving scanning efficiency and effectiveness. In addition, controlling the pause and restart of the scanning process according to an actual situation allows for personalized management of the scanning process.

FIG. 4 is a flowchart of an exemplary process for determining an activation position according to some embodiments of the present disclosure. In some embodiments, magnetic resonance scanning may be performed by one or more components (e.g., the processing device 120) of the system 100 for magnetic resonance scanning or one or more modules (e.g., the activation module 220) of the system 200 for magnetic resonance scanning. For example, process 400 may be stored in a storage device (e.g., the storage device 150) in the form of a program or commands, and when the processing device 120 or the activation module 220 executes the program or the commands, process 400 may be implemented. The descriptions of the operations of process 400 provided below are illustrative. In some embodiments, one or more additional operations not described and/or one or more operations not discussed may be used to complete the process 400. In addition, the sequence of operations of process 400 shown in FIG. 4 and described below is not intended to be limiting.

In 401, a trigger state of a protocol sequence suspending command may be determined

In some embodiments, the activation module 220 may determine whether the trigger state of the protocol sequence suspending command is a first trigger state or a second trigger state, and perform subsequent operations based on a determination result. In some embodiments, the activation module 220 may determine the trigger state of the protocol sequence suspending command based on a suspending state of a scanning sequence. For example, if the scanning sequence is in a state of active suspension, the activation module 220 may determine that the trigger state of the protocol sequence suspending command is the first trigger state; if the scanning sequence is in a state of abnormal suspension, the activation module 220 may determine that the trigger state of the protocol sequence suspending command is the second trigger state.

It should be noted that after determining the trigger state of the protocol sequence suspending command based on operation 401, either operations 402-403 or operations 404-405 may be performed. In other words, the order between operations 402-403 and operations 404-405 is not restricted.

In 402, in response to determining that the trigger state is the first trigger state, a first suspension position of a first scanning task may be determined.

The first scanning task refers to a currently executing scanning task recorded when the scanning sequence is suspended. In some embodiments, the first scanning task may be a currently executing scanning task recorded when the scanning sequence is actively suspended. For example, if the scanning sequence is actively suspended, a preceding completed scanning task is Scanning Task 1, and a currently executing scanning task is Scanning Task 2, then Scanning Task 2 may be determined as the first scanning task.

In some embodiments, when the scanning sequence is suspended, the scanning state of the first scanning task may be completed or being executed. If the first scanning task is completed, the first scanning task and a raw data file corresponding to the first scanning task may be recorded. If the first scanning task is being executed, an interruption position and a scanning state of the first scanning task may be recorded. For example, if Scanning Task 2 is the first scanning task, when scanning task 2 is completed, Scanning Task 2 and a raw data file corresponding to Scanning Task 2 may be recorded; and when Scanning Task 2 is being executed, a scanning situation of Scanning Task 2 may be recorded. The scanning situation of Scanning Task 2 may include that the interruption position is a “position point A” and the scanning state is “being executed.” More descriptions of the interruption position may be found in operation 303 and the related descriptions thereof, which will not be reiterated here.

The first suspension position refers to an ending position of the first scanning task when the scanning sequence is suspended. In some embodiments, the first suspension position may be related to the scanning state of the first scanning task. In some embodiments, when the first scanning task is in completed, the first suspension position may be an endpoint position of the first scanning task. The endpoint position refers to an ending position when the entire scanning task is completed. In some embodiments, when the first scanning task is being executed, the first suspension position may be the interruption position of the first scanning task.

In some embodiments, in response to determining that the trigger state is the first trigger state, the activation module 220 may determine the first suspension position of the first scanning task based on the scanning state of the first scanning task.

In some embodiments, when the first scanning task is completed, the activation module 220 may determine the endpoint position of the first scanning task as the first suspension position. For example, if the first scanning task is completed and its endpoint position is “position point A,” then the position point A may be determined as the first suspension position.

In some embodiments, when the first scanning task is being executed, the activation module 220 may determine the interruption position of the first scanning task as the first suspension position. For example, if the first scanning task is being executed and its interruption position is “position point B,” then position point B may be determined as the first suspension position.

In 403, the first suspension position may be determined as the activation position.

In some embodiments, in response to determining that the trigger state is the first trigger state, the activation module 220 may determine the first suspension position as the activation position. In some embodiments, the activation module 220 may continue to perform the subsequent scanning of the scanning sequence based on the activation position. More descriptions of continuing to perform the subsequent scanning may be found in operation 306 and the related descriptions thereof, which will not be reiterated here.

In 404, in response to determining that the trigger state is the second trigger state, a second suspension position of a second scanning task may be determined.

The second scanning task refers to a preceding completed scanning task recorded when the scanning sequence is suspended. In some embodiments, the second scanning task may be the preceding completed scanning task recorded when the scanning sequence is abnormally suspended. For example, when the scanning sequence is abnormally suspended, the preceding completed scanning task is Scanning Task 1, and the currently executing scanning task is Scanning Task 2, then Scanning Task 1 may be determined as the second scanning task.

In some embodiments, when the scanning sequence is suspended, and the second scanning task is completed, the second scanning task and a raw data file corresponding to the second scanning task may be recorded. More descriptions of recording a scanning task and a raw data file corresponding to the second scanning task may be found in operation 303 and the related descriptions thereof.

The second suspension position refers to an ending position of the second scanning task when the scanning sequence is suspended. In some embodiments, the second suspension position may be an endpoint position of the second scanning task.

In some embodiments, in response to determining that the trigger state is the second trigger state, the activation module 220 may determine the endpoint position of the second scanning task as the second suspension position. For example, if the endpoint position of the second scanning task is “position point A,” then the position point A may be determined as the second suspension position.

In 405, the second suspension position may be determined as the activation position.

In some embodiments, in response to determining that the trigger state is the second trigger state, the activation module 220 may determine the second suspension position as the activation position. In some embodiments, the activation module 220 may continue to perform the subsequent scanning of the scanning sequence based on the activation position. More descriptions of continuing to perform the subsequent scanning may be found in operation 306 and the related descriptions thereof, which will not be reiterated here.

In some embodiments described in the present disclosure, based on the trigger state of the protocol sequence suspending command, the activation position for resuming the subsequent scanning may be determined. If the trigger state of the protocol sequence suspending command is the first trigger state, it may indicate that the scanning sequence is suspended by the user's manual action and no abnormalities occurs in a magnetic resonance device during the scanning process. In such cases, the magnetic resonance device may resume the subsequent scanning from where it stopped to ensure the coherence, accuracy, and efficiency of scanning data to a maximum extent. If the trigger state of the protocol sequence suspending command is the second trigger state, it may indicate an abnormal suspension of the scanning sequence, possibly due to an abnormality or a crash during the execution of the scanning task. In such cases, the magnetic resonance device may discard incomplete raw data of the scanning task and resume the subsequent scanning from a starting point of the scanning task to ensure reliability of the scanning data and effectively avoid the wastage of previous scanning data, thereby enhancing the scanning efficiency.

It should be noted that the descriptions of process 300 and process 400 above are merely illustrative and explanatory, not limiting the scope of the present disclosure. For those skilled in the art, various modifications and changes may be made to process 300 and process 400 under the guidance of the present disclosure. However, these modifications and changes are still within the scope of the present disclosure.

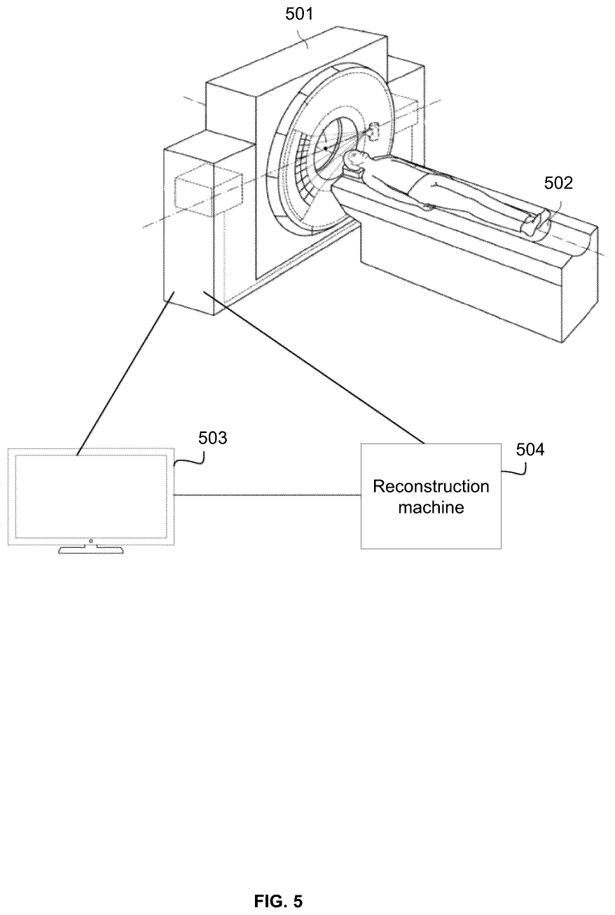

FIG. 5 is a schematic diagram of an exemplary application scenario of magnetic resonance scanning according to some embodiments of the present disclosure. As shown in FIG. 5, the application scenario may include a system for magnetic resonance scanning, which may include: a magnetic resonance device 501, a scanning bed 502, a host 503, and a reconstruction machine 504. An operator may control the magnetic resonance device 501 via the host 503 to perform layered scanning on a target object on the scanning bed 502 to obtain a large amount of raw data files. The large amount of raw data files may be stored in corresponding storage spaces according to different data slicing rules, so that the reconstruction machine 504 may retrieve the corresponding raw data from the corresponding storage space for image reconstruction. In some embodiments, the system for magnetic resonance scanning may be a system for magnetic resonance imaging of animals. The magnetic resonance device may obtain massive primary data by performing magnetic resonance scanning on an object, especially when scanning an animal for preclinical life science research. The massive primary data may be reconstructed into digital images for display. The massive primary data without any processing may be defined as raw data. It may be understood that during the process for performing magnetic resonance scanning on the object using the magnetic resonance device, in addition to obtaining the raw data, noise data may also be obtained. The raw data may include the noise data.

In one embodiment, as shown in FIG. 6A, an exemplary process for data storage in magnetic resonance scanning is provided. Taking the process applied to the system for magnetic resonance scanning in FIG. 5 as an example to illustrate, the process may include one or more of the following operations.

In 601, a raw data file of a current scanning layer may be obtained during scanning a target object using the magnetic resonance device.

In some embodiments, when scanning the target object, the magnetic resonance device may divide the target object into one or more scanning layers and scan each of the scanning layers. In some embodiments, the magnetic resonance device may perform the scanning in an order of the divided scanning layers or not in the order of the divided scanning layers, which is not limited here. During the scanning process, the raw data file of the current scanning layer may be obtained.

In 602, a target file group corresponding to the raw data file of the current scanning layer may be determined according to a preset data slicing rule.

The data slicing rule is used to group raw data files. The preset data slicing rule may include at least one of: a first rule for grouping raw data files based on a layer count, a second rule for grouping raw data files based on a scanning time, a third rule for grouping raw data files based on a data capacity, a fourth rule for grouping raw data files based on a count of repetitions when repeatedly scanning a scanning sequence or a scanning layer, a fifth rule for grouping raw data files generated by scanning in different scan directions in different scanning sequences, or other rules for grouping raw data files that may support the sequence scanning.

In some embodiments, determining the target file group corresponding to the raw data file of the current scanning layer according to the preset data slicing rule may include dividing all scanning layers into one or more groups. For example, when scanning layers in a first preset group are scanned, raw data files generated by a current scanning layer during scanning in the scanning layers may be grouped according to the first rule; and when scanning layers in other groups are scanned, raw data files generated by a current scanning layer during scanning in the scanning layers may be grouped according to the second rule or the third rule. As another example, when the scanning layers in the first preset group are scanned, the raw data files generated by the current scanning layer during scanning in the scanning layers may be grouped according to the third rule; and when the scanning layers in other groups are scanned, the raw data files generated by the current scanning layer during scanning in the scanning layers may be grouped according to the first rule or the second rule. As a further example, when all the scanning layers are scanned, raw data files generated by each of the scanning layers during scanning may be grouped according to any of the preset data slicing rules. As a further example, all the scanning layers may be divided into three groups, a target file group of raw data files of a current scanning layer in the three groups may be determined based on different data slicing rules, respectively. Merely by way of an example, 12 scanning layers may be divided into three groups (scanning layers 1-4 as a first group, scanning layers 5-8 as a second group, and scanning layers 9-12 as a third group), a target file group of raw data files of a current scanning layer generated during scanning in the first group of scanning layers may be determined according to the first rule, a target file group of raw data files of a current scanning layer generated during scanning in the second group of scanning layers may be determined according to the second rule, and a target file group of raw data files of a current scanning layer generated during scanning in the third group of scanning layers may be determined according to the third rule.

In some embodiments, when it is necessary to repeatedly scanning a scanning sequence or a scanning layer, the raw data files generated from each scanning may be grouped according to the fifth rule for grouping raw data files based on the count of repetitions. For example, the raw data files generated from scanning with odd repetition counts may be grouped and stored in a corresponding storage space, and the raw data files generated from scanning with even repetition counts may be grouped and stored in a corresponding storage space. As another example, raw data files generated from scanning with specific repetition counts may be divided into a group, which is not limited here.

In some embodiments, a user may select the data slicing rule based on a raw data slice selection interface shown in FIG. 6B.

In 603, data of the raw data file of the current scanning layer may be stored into a storage space corresponding to the target file group.

In some embodiments, storing the data of the raw data file of the current scanning layer into the storage space corresponding to the target file group may include storing the data of the raw data file of the current scanning layer in the storage space corresponding to the target file group, or packaging the data of the raw data file of the current scanning layer and storing the packaged data in the storage space corresponding to the target file group, which is not limited by the present disclosure.

In the above process for data storage in magnetic resonance scanning, by obtaining the raw data file of the current scanning layer during scanning the target object using the magnetic resonance device, the target file group corresponding to the raw data file of the current scanning layer may be determined according to the preset data slicing rule, and data of the raw data file of the current scanning layer may be stored into the storage space corresponding to the target file group, which make it possible to store raw data files generated by different scanning layers into the target file groups corresponding to the raw data files, respectively, thereby facilitating rapid and accurate retrieval of corresponding primary medical imaging data for image reconstruction during subsequent processes.

In one embodiment, determining the target file group corresponding to the raw data file of the current scanning layer according to the preset data slicing rule may include a following process.

The process may include: determining the target file group corresponding to the raw data file of the current scanning layer based on a scanning layer identifier of the current scanning layer according to a first rule, wherein the first rule may include a correspondence between scanning layer identifiers and file groups.

In some embodiments, the correspondence between the scanning layer identifiers and the file groups may include determining the target file group corresponding to the raw data file of the current scanning layer when the scanning layer identifier is a preset layer count. The preset layer count may be a continuous preset layer count or a discontinuous preset layer count, without limitation here. When scanning of the current scanning layer is completed, a raw data file may be generated. At this point, based on the identifier of the current scanning layer, the layer count of the current scanning layer may be determined. Based on the correspondence between scanning layer identifiers and file groups, the target file group corresponding to the raw data file of the current scanning layer may be determined. For example, for a total of 12 scanning layers, if the scanning layer identifier of the current scanning layer is a first layer, a second layer, a third layer, or a fourth layer, the target file group corresponding to the raw data file of the current scanning layer may be determined as a first group; if the scanning layer identifier of the current scanning layer is a fifth layer, a sixth layer, a seventh layer, or an eighth layer, the target file group corresponding to the raw data file of the current scanning layer may be determined as a second group; and if the scanning layer identifier of the current scanning layer is a ninth layer, a tenth layer, an eleventh layer, or a twelfth layer, the target file group corresponding to the raw data file of the current scanning layer may be determined as a third group. As another example, for a total of 12 scanning layers, if the scanning layer identifier of the current scanning layer is a first layer, a third layer, a fifth layer, or a seventh layer, the target file group corresponding to the raw data file of the current scanning layer may be determined as a first group; if the scanning layer identifier of the current scanning layer is a second layer, a fourth layer, a sixth layer, or an eighth layer, the target file group corresponding to the raw data file of the current scanning layer may be determined as a second group; and if the scanning layer identifier of the current scanning layer is a ninth layer, a tenth layer, an eleventh layer, or a twelfth layer, the target file group corresponding to the raw data file of the current scanning layer may be determined as a third group.

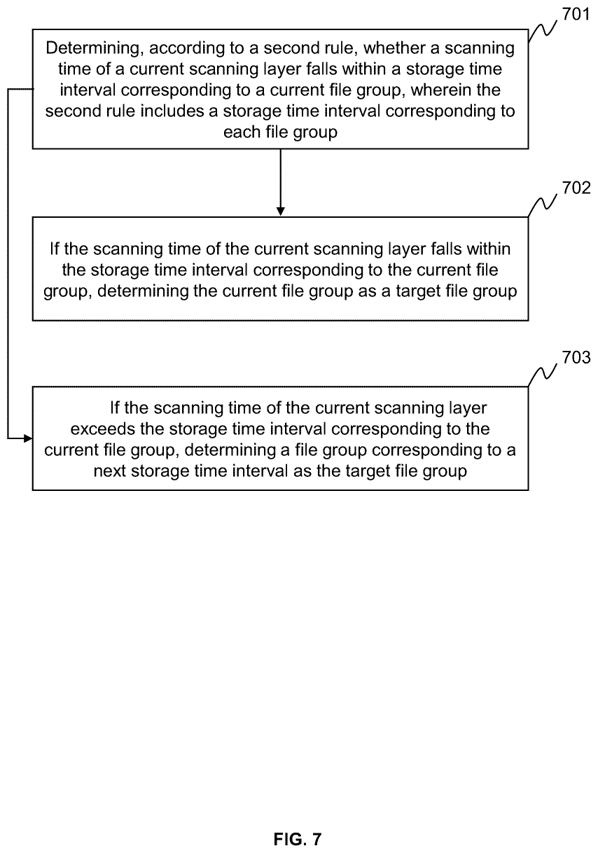

In one embodiment, as shown in FIG. 7, a process for determining the target file group corresponding to the raw data file of the current scanning layer according to the preset data slicing rule may include following operations.

In 701, whether a scanning time of the current scanning layer falls within a storage time interval corresponding to a current file group may be determined according to a second rule, wherein the second rule includes a storage time interval corresponding to each file group.

In some embodiments, since the target file group is determined based on a distribution of scanning times for the current scanning layer, for example, if a total scanning time of the magnetic resonance device is 30 minutes, the first 10 minutes of the scanning time may correspond to a first storage time interval, the middle 10 minutes of the scanning time may correspond to a second storage time interval, and the last 10 minutes of the scanning time may correspond to a third storage time interval. Each storage time interval may correspond to a different target file group, e.g., the first storage time interval corresponds to a first target file group, the second storage time interval corresponds to a second target file group, and the third storage time interval corresponds to a third target file group. Initially, the first target file group may be designated as the current file group, and when data storage of the first target file group is completed, the second target file group may be designated as the current file group, and so on. According to the second rule, it may be determined whether the scanning time of the current scanning layer falls within the storage time interval corresponding to the current file group. For example, if the scanning time of the current scanning layer is at the 17th minute, it may be assumed that the raw data file for the first 10 minutes have been stored in the first target file group. Then, it may be determined whether the scanning time of the current scanning layer falls within the storage time interval corresponding to the second target file group. If the scanning time of the current scanning layer falls within the storage time interval corresponding to the second target file group, the raw data file of the current scanning layer corresponds to the second target file group. If the scanning time of the current scanning layer exceeds the storage time interval of the second target file group, it may be determined whether the scanning time of the current scanning layer falls within the storage time interval corresponding to the third target file group. If the scanning time of the current scanning layer falls within the storage time interval corresponding to the third target file group, the raw data file of the current scanning layer corresponds to the third target file group. If the scanning time of the current scanning layer exceeds the storage time interval corresponding to the third target file group, the third target file group may be determined as the target file group. Alternatively, if the scanning time of the raw data file of the current scanning layer falls within the storage time interval corresponding to the current file group, the current file group may be determined as the target file group. If the scanning time of the raw data file of the current scanning layer exceeds the storage time interval corresponding to the current file group, a file group corresponding to a next storage time interval may be determined as the target file group.

In 702, if the scanning time of the current scanning layer falls within the storage time interval corresponding to the current file group, the current file group may be determined as the target file group.

In some embodiments, if the scanning time of the current scanning layer falls within the storage time interval corresponding to the current file group, the current file group may be determined as the target file group. For example, if the scanning time of the current scanning layer is at the 9th minute, which falls within the first time period, then it may be determined that the raw data file of the current scanning layer corresponds to the first target file group.

In 703, if the scanning time of the current scanning layer exceeds the storage time interval corresponding to the current file group, a file group corresponding to a next storage time interval may be determined as the target file group.

In some embodiments, if the scanning time of the current scanning layer exceeds the storage time interval corresponding to the current file group, the file grouping corresponding to the next storage time interval may be determined as the target file group. For example, if the scanning time of the current scanning layer is at the 11th minute, exceeding the storage time interval of the first target file group, then the file group corresponding to the second storage time interval may be determined as the target file group.

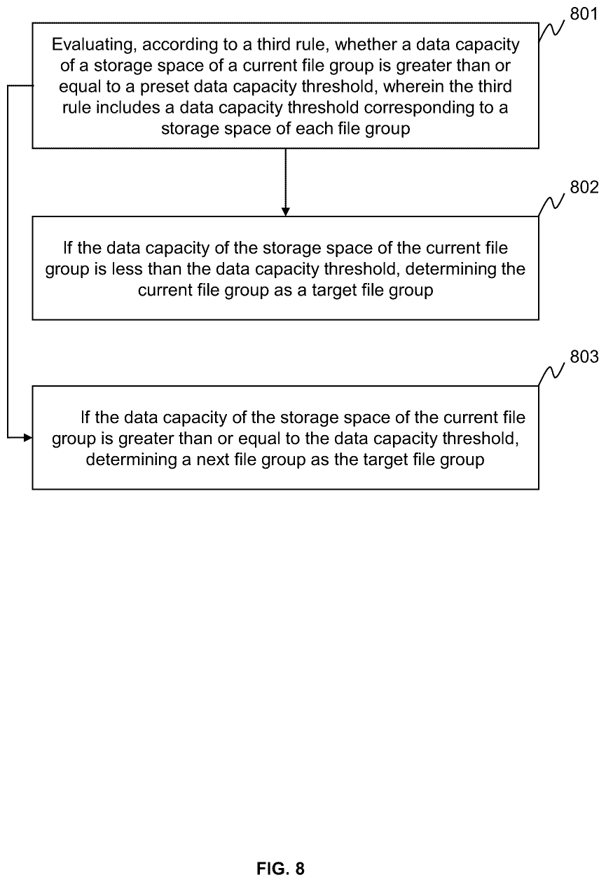

In one embodiment, as shown in FIG. 8, determining the target file group corresponding to the raw data file of the current scanning layer according to the preset data slicing rule may include following operations.

In 801, whether a data capacity of a storage space of a current file group is greater than or equal to a preset data capacity threshold may be evaluated according to a third rule, wherein the third rule may include a data capacity threshold corresponding to a storage space of each file group.

In 802, if the data capacity of the storage space of the current file group is less than the data capacity threshold, the current file group may be determined as the target file group.

In 803, if the data capacity of the storage space of the current file group is greater than or equal to the data capacity threshold, a next file group may be determined as the target file group.

In some embodiments, after the raw data file is generated from the current scanning layer, it may be determined whether the data capacity of the current file group is greater than or equal to the preset data capacity threshold. If the data capacity of the storage space of the current file group is less than the data capacity threshold, the current file group may be determined as the target file group. If the data capacity of the storage space of the current file group is greater than or equal to the data capacity threshold, the next file group may be determined as the target file group. The data capacities corresponding to storage spaces of different file groups may be the same or different. The third rule may include a data capacity threshold corresponding to the storage space of each file group, wherein the data capacity threshold may indicate whether the storage space of the file group may accommodate additional data. For example, if the data capacity of the storage space of the current file group has reached 20 G and the preset data capacity threshold is 25 G, the current file group may be determined as the target file group. If the data capacity of the storage space of the current file group has reached 20 G and the preset data capacity threshold is also set to 20 G, it indicates that the current file group may not accommodate additional data, and therefore, the next file group may be determined as the target file group.

In some embodiments, the target file group corresponding to the raw data file of the current scanning layer may be determined according to the three different rules to facilitate storing the raw data file in the corresponding target file group, ensuring quick and accurate retrieval during a subsequent reconstruction process.

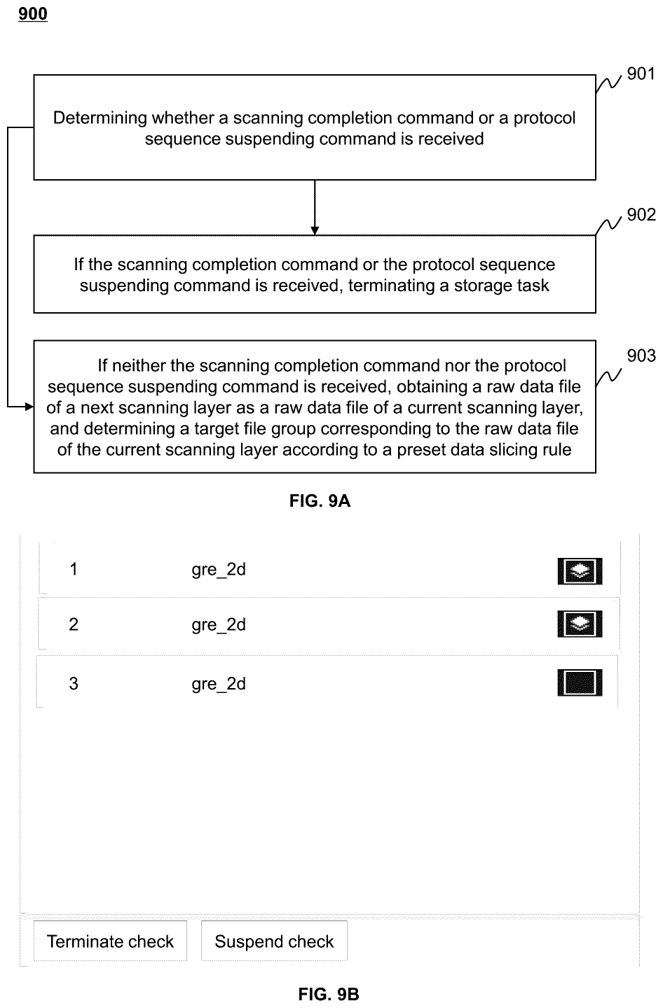

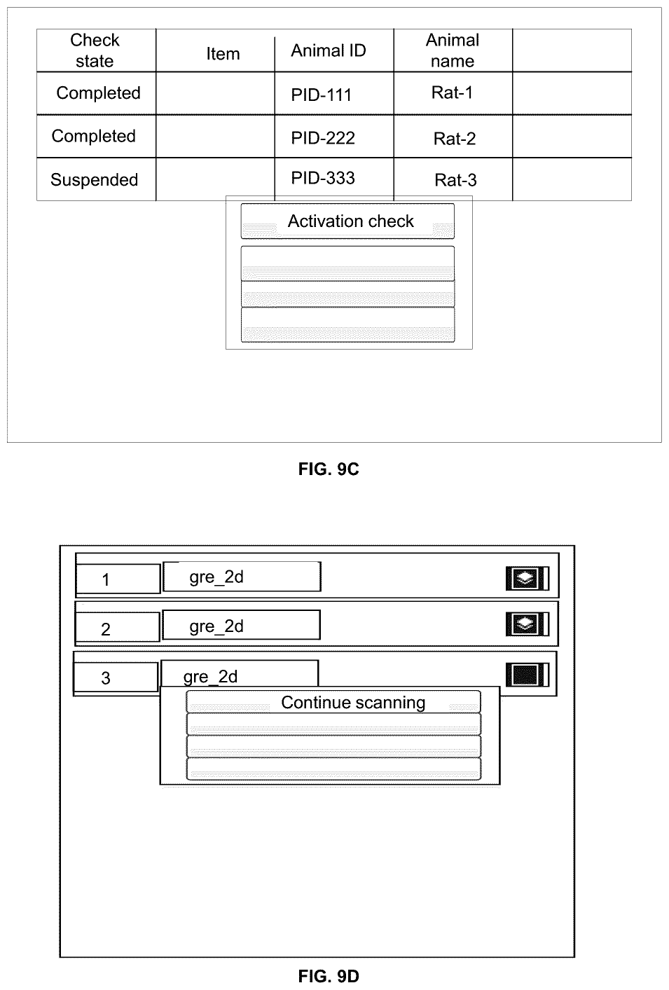

The above embodiments have described how to determine the target file group for storing raw data. During scanning using the magnetic resonance device, a protocol sequence suspending command may be received, which is described below using one embodiment as an example. In one embodiment, as shown in FIG. 9A, the process for data storage in magnetic resonance scanning may further include one or more of the following operations.

In 901, whether a scanning completion command or a protocol sequence suspending command is received may be determined.

In 902, if the scanning completion command or the protocol sequence suspending command is received, a storage task may be terminated.

In 903, if neither the scanning completion command nor the protocol sequence suspending command is received, a raw data file of a next scanning layer may be obtained as the raw data file of the current scanning layer, and the target file group corresponding to the raw data file of the current scanning layer may be determined according to the preset data slicing rule.

In some embodiments, when the scanning is completed, a scanning completion command may be generated accordingly. If the scanning completion command is received, the storage task may be terminated. During scanning using the magnetic resonance device, a user may click a suspend check button on a protocol scanning interface, as shown in FIG. 9B, to suspend a current scanning sequence and generate a protocol sequence suspending command. If the protocol sequence suspending command is received, the data storage task may be terminated. If neither the scanning completion command nor the protocol sequence suspending command is received during the scanning of the current scanning layer, the raw data file of the next scanning layer may be obtained as the raw data file for the current scanning layer, and the operations for determining the target file group corresponding to the raw data file of the current scanning layer according to the preset data slicing rule may be performed.

In some embodiments, in response to receiving the protocol sequence suspending command, the magnetic resonance device may be controlled to stop scanning the target object, a layer count of scanning layers that have been scanned may be recorded, and a task execution log file may be generated.

The protocol sequence suspending command refers to a command sent by the user to suspend the scanning or a suspending command triggered by system abnormal termination during the scanning.

In some embodiments, according to a scanning sequence resume interface as shown in FIG. 9C, the user may identify a suspended scanning sequence that needs to be resumed and select a resume option. At this point, the interface may be navigated to a checklist interface, which may be suspended over a position of the suspended scanning sequence that needs to be resumed, with the checklist interface located above a row corresponding to a selected sequence item. As shown in FIG. 9D, the user may select a resume scanning option to continue the protocol scanning and trigger a suspending protocol sequence activation command. In response to receiving the suspending protocol sequence activation command, the task execution log file may be searched for, the layer count of scanning layers that have been scanned may be retrieved, and layers that have not been scanned may be scanned.

In some embodiments, before the user triggers the protocol sequence suspending command by clicking the suspend check button on the protocol scanning interface, the user may be prompted to decide to discard the data of the current scanning layer or wait for a completion of a current acquisition cycle to suspend the check. If the user chooses to discard the data of the current scanning layer, the raw data of the current scanning layer may not be stored, the protocol sequence suspending command may be triggered, and the current scanning layer may not be recorded.

In this embodiment, whether the scanning completion command or the protocol sequence suspending command is received may be determined, if the scanning completion command or the protocol sequence suspending command is received, the storage task may be terminated. If neither the scanning completion command or the protocol sequence suspending command is not received, the raw data file of the next scanning layer may be obtained as the raw data file of the current scanning layer, and the operations for determining the target file group corresponding to the raw data file of the current scanning layer according to the preset data slicing rule may be performed. This enables real-time termination of the storage task based on the scanning completion command or the protocol sequence suspending command and the ability to restart scanning when receiving the suspending protocol sequence activation command, addressing the inability to suspend operations in existing technologies and enhancing user experience.

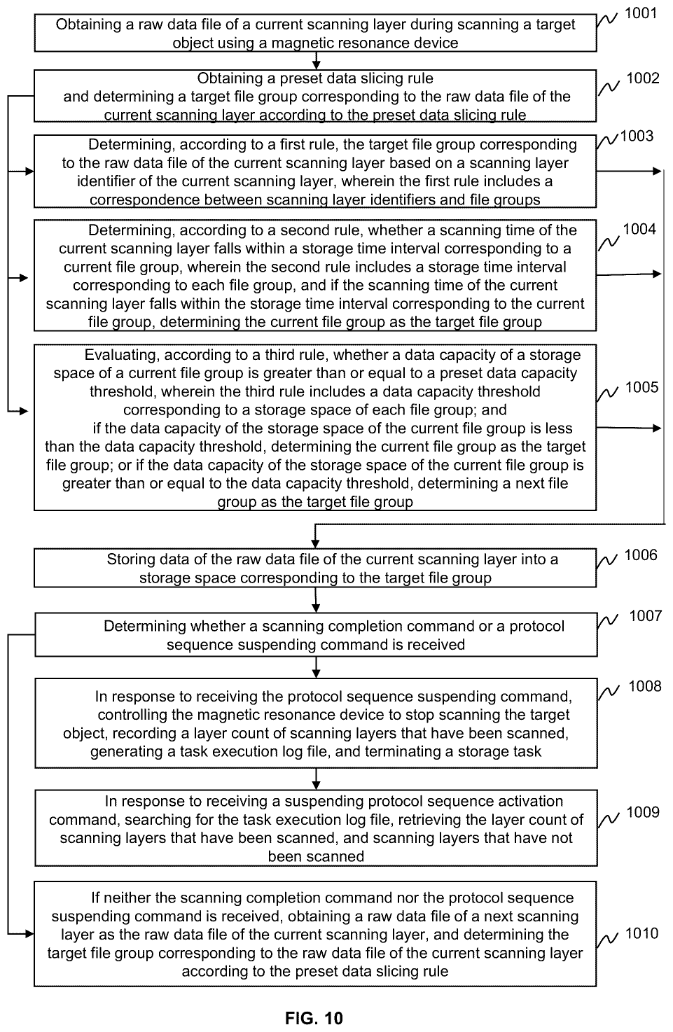

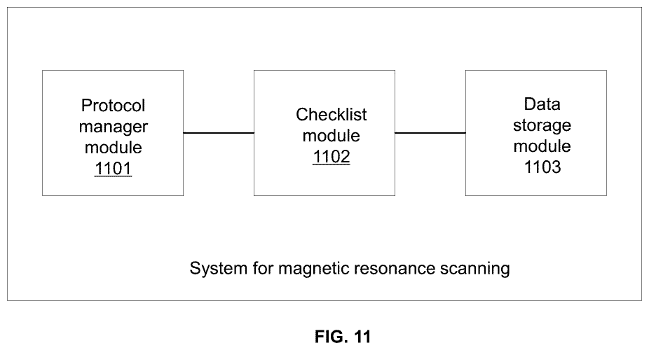

In order to facilitate the understanding of those skilled in the art, an embodiment is described below to further illustrate the process for data storage in magnetic resonance scanning. In one embodiment, as shown in FIG. 10, the process for data storage in magnetic resonance scanning may include one or more of the following operations.

In 1001, a raw data file of a current scanning layer may be obtained during scanning a target object using a magnetic resonance device.

In 1002, a preset data slicing rule may be obtained and a target file group corresponding to the raw data file of the current scanning layer may be determined according to the preset data slicing rule.

In 1003, the target file group corresponding to the raw data file of the current scanning layer may be determined based on a scanning layer identifier of the current scanning layer according to a first rule, wherein the first rule may include a correspondence between scanning layer identifiers and file groups.

In 1004, whether a scanning time of the current scanning layer falls within a storage time interval corresponding to a current file group may be determined according to a second rule, wherein the second rule may include a storage time interval corresponding to each file group. If the scanning time of the current scanning layer falls within the storage time interval corresponding to the current file group, the current file group may be determined as the target file group.

In 1005, whether a data capacity of a storage space of the current file group is greater than or equal to a preset data capacity threshold may be evaluated according to a third rule, wherein the third rule may include a data capacity threshold corresponding to a storage space of each file group. If the data capacity of the storage space of the current file group is less than the data capacity threshold, the current file group may be determined as the target file group; or if the data capacity of the storage space of the current file group is greater than or equal to the data capacity threshold, a next file group may be determined as the target file group.

In 1006, data of the raw data file of the current scanning layer may be stored into the storage space corresponding to the target file group.

In 1007, whether a scanning completion command or a protocol sequence suspending command is received may be determined.

In 1008, in response to receiving the protocol sequence suspending command, a magnetic resonance device may be controlled to stop scanning a target object, a layer count of scanning layers that have been scanned may be recorded, a task execution log file may be generated, and a storage task may be terminated.

In 1009, in response to receiving a suspending protocol sequence activation command, the task execution log file may be searched for, the layer count of scanning layers that have been scanned may be retrieved, and layers that have not been scanned may be scanned.

In 1010, if neither the scanning completion command nor the protocol sequence suspending command is received, a raw data file of a next scanning layer may be obtained as the raw data file for the current scanning layer, and the target file group corresponding to the raw data file of the current scanning layer may be determined according to the preset data slicing rule.

In this embodiment, during the scanning of the target object using the magnetic resonance device, the raw data file of the current scanning layer may be obtained. According to the preset data slicing rule, the target file group corresponding to the raw data file of the current scanning layer may be determined. The data of the raw data file of the current scanning layer may be then stored in the storage space corresponding to the target file group. In this approach, the raw data files generated from scanning different scanning layers may be stored in the corresponding target file groups, respectively, facilitating rapid and accurate retrieval of the corresponding original medical image data for image reconstruction in subsequent processes.

It should be understood that although the operations in the processes of FIGS. 6A-10 are shown sequentially according to the arrows, the operations are not necessarily executed sequentially as indicated by the arrows. Unless otherwise specified in the present disclosure, the execution of the operations is not strictly limited to a particular order, and the operations may be performed in a different order. Moreover, at least some of the operations in FIGS. 6A-10 may include multiple sub-operations or stages, which may not necessarily be completed at the same time but may be executed at different times. The execution order of these sub-operations or stages is also not necessarily sequential, but may occur alternately or interchangeably with at least some sub-operations in other operations or stages.

The above-described embodiments illustrate the method for data storage in magnetic resonance scanning. Below, an embodiment of a system for magnetic resonance scanning using the method for data storage in magnetic resonance scanning is described. In one embodiment, as shown in FIG. 11, the system for magnetic resonance scanning may include: a protocol manager module 1101, a checklist module 1102, and a data storage module 1103.

The protocol manager module 1101 may be configured to obtain a target scanning protocol selected by a user via a protocol scanning interface and transmit the target scanning protocol to the checklist module.

The checklist module 1102 may be configured to navigate to a slice selection interface based on the target scanning protocol, obtain a preset data slicing rule selected by the user via the slice selection interface, and scan a target object to generate a raw data file of a current scanning layer.

The data storage module 1103 may be configured to store the raw data file of the current scanning layer according to the preset data slicing rule.

In some embodiments, when performing the magnetic resonance scanning, the user may select a desired target scanning protocol option based on a protocol scanning interface. The protocol manager module 1101 may obtain the target scanning protocol and transmits the selected target scanning protocol to the checklist module 1102. The user may open the target scanning protocol by clicking, and the checklist module 1102 may generate a protocol parameter card based on the target scanning protocol. The user may select a count of coils based on a protocol parameter card and navigate to a slice selection interface. The protocol manager module 1101 may obtain the preset data slicing rule selected by the user and scan the target object to generate the raw data file of the current scanning layer. The data storage module 1103 may then store the raw data file of the current layer according to the preset data slicing rule. The data slicing rule is used to group raw data files.

In some embodiments, the data slicing rule may include at least one of: a first rule for grouping raw data files based on a layer count, a second rule for grouping raw data files based on a scanning time, or a third rule for grouping raw data files based on a data capacity.

More descriptions of the data storage module may be found in the descriptions of the process for data storage in magnetic resonance scanning above. The modules in the system for magnetic resonance scanning described above may be fully or partially implemented through software, hardware, or a combination thereof. The modules may be embedded in or independent of at least one processor in an electronic device in hardware form, or stored in memory in software form for the at least one processor to call and execute one or more operations corresponding to each module.

In this embodiment, the system for magnetic resonance scanning includes the protocol manager module, the checklist module, and the data storage module. The protocol manager module may be configured to obtain the target scanning protocol selected by the user via the protocol scanning interface and transmit the target scanning protocol to the checklist module. The checklist module may be configured to navigate to the slice selection interface based on the target scanning protocol, obtain the preset data slicing rule selected by the user via the slice selection interface, and scan the target object to generate the raw data file of the current scanning layer. The data storage module may be configured to store the raw data file of the current scanning layer according to the preset data slicing rule. The data slicing rule is used to group raw data files. By providing the user with different data slicing rules, the raw data files of scanning layers can be grouped to the target file groups respectively, thereby enabling quick and accurate retrieval of corresponding data content during subsequent data reconstruction.

In one embodiment, the system for magnetic resonance scanning may further include a suspending module and an activation module.

The suspending module may be configured to obtain a protocol sequence suspending command based on the protocol scanning interface and when the protocol sequence suspending command is received, control a magnetic resonance device to stop scanning the target object, record a layer count of scanning layers that have been scanned, generate a task execution log file, and transmit the task execution log file to the data storage module for storage.

The activation module may be configured to obtain a suspending protocol sequence activation command based on the protocol scanning interface, search for the task execution log file in the data storage module, retrieve the layer count of scanning layers that have been scanned, and scanning layers that have not been scanned.

More descriptions of the suspending module and activation module may be found in the descriptions of the process for data storage in magnetic resonance scanning mentioned above and are not reiterated here.

In this embodiment, the suspending module may obtain the protocol sequence suspending command based on the protocol scanning interface, and when receiving the protocol sequence suspending command, control the magnetic resonance device to stop scanning the target object. The suspending module may then record the layer count of scanning layers that have been scanned, generate the task execution log file, and transmit the task execution log file to the data storage module for storage. The activation module obtain the suspending protocol sequence activation command based on the protocol scanning interface, search for the task execution log file in the data storage module, retrieve the layer count of scanning layers that have been scanned, and scan layers that have not been scanned. Through the configurations of the suspending module and the activation module, real-time termination of the storage task can be achieved based on the scanning completion command or the protocol sequence suspending command and scanning can be resumed when the protocol sequence suspending activation command is received, thereby addressing the inability to suspend operations in existing technologies and enhancing user experience.

In one embodiment, the data storage module may be configured to determine, according to a first rule, the target file group corresponding to the raw data file of the current scanning layer based on a scanning layer identifier of the current scanning layer, wherein the first rule may include a correspondence between scanning layer identifiers and file groups.

In one embodiment, the data storage module may be configured to determine, according to a second rule, whether a scanning time of the current scanning layer falls within a storage time interval corresponding to a current file group, wherein the second rule may include a storage time interval corresponding to each file group. If the scanning time of the current scanning layer falls within the storage time interval corresponding to the current file group, the data storage module may determine the current file group as the target file group; or if the scanning time of the current scanning layer exceeds the storage time interval corresponding to the current file group, the data storage module may determine a file group corresponding to a next storage time interval as the target file group.

In one embodiment, the data storage module may be configured to evaluate, according to a third rule, whether a data capacity of a storage space of a current file group is greater than or equal to a preset data capacity threshold, wherein the third rule may include a data capacity threshold corresponding to a storage space of each file group. If the data capacity of the storage space of the current file group is less than the data capacity threshold, the data storage module may determine the current file group as the target file group; or if the data capacity of the storage space of the current file group is greater than or equal to the data capacity threshold, the data storage module may determine a next file group as the target file group.

In one embodiment, the data storage module may be configured to determine whether a scanning completion command or a protocol sequence suspending command is received. If the scanning completion command or the protocol sequence suspending command is received, the data storage module may terminate a storage task. If neither the scanning completion command nor the protocol sequence suspending command is received, the data storage module may obtain a raw data file of a next scanning layer as the raw data file of the current scanning layer, and determine the target file group corresponding to the raw data file of the current scanning layer according to the preset data slicing rule.

In one embodiment, the data storage module may be configured to: in response to receiving the protocol sequence suspending command, control the magnetic resonance device to stop scanning the target object, record a layer count of scanning layers that have been scanned, and generate a task execution log file.

In one embodiment, the data storage module may be configured to: in response to receiving a suspending protocol sequence activation command, search for the task execution log file, retrieve the layer count of scanning layers that have been scanned, and scan layers that have not been scanned.

The descriptions of the data storage module may be found in the descriptions of the process for data storage in magnetic resonance scanning mentioned above, and are not reiterated here.

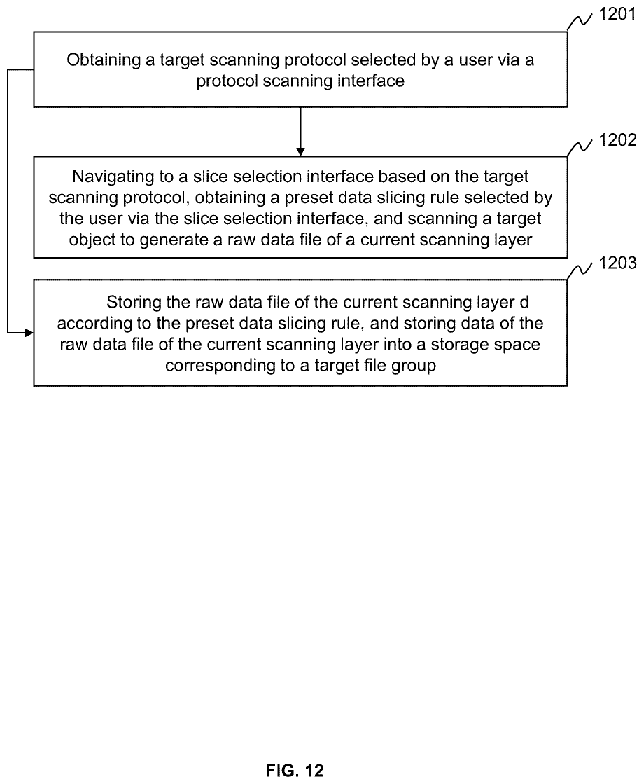

The above embodiments have described the process for data storage in magnetic resonance scanning after scanning using the magnetic resonance device. Below, an embodiment is provided to describe a process for magnetic resonance scanning. In one embodiment, as shown in FIG. 12, the process for magnetic resonance scanning may include the following operations.

In 1201, a target scanning protocol selected by a user via a protocol scanning interface may be obtained.

In 1202, a slice selection interface may be navigated to based on the target scanning protocol, a preset data slicing rule selected by the user via the slice selection interface may be obtained, and a target object may be scanned to generate a raw data file of a current scanning layer.

In 1203, the raw data file of the current scanning layer may be stored according to the preset data slicing rule, and data of the raw data file of the current scanning layer may be stored into a storage space corresponding to a target file group.

More descriptions regarding magnetic resonance scanning may be found in the descriptions of the system for magnetic resonance scanning above, which will not be reiterated here.

In some embodiments, the data slicing rule may include at least one of: a first rule for grouping raw data files based on a layer count, a second rule for grouping raw data files based on a scanning time, or a third rule for grouping raw data files based on a data capacity.

More descriptions of operation 1203 may be found in the descriptions of the process for data storage in magnetic resonance scanning above, which will not be reiterated here.

In this embodiment, the target scanning protocol selected by the user via a protocol scanning interface may be obtained. The slice selection interface may be navigated to based on the target scanning protocol, the preset data slicing rule selected by the user via the slice selection interface may be obtained, and the target object may be scanned to generate the raw data file of the current scanning layer. The raw data file of the current scanning layer may be stored according to the preset data slicing rule. The data slicing rule is used to group raw data files. By providing the user with different data slicing rules, the raw data files of scanning layers can be grouped to the target file groups respectively, thereby enabling quick and accurate retrieval of corresponding data content during subsequent data reconstruction.

In one embodiment, determining the target file group corresponding to the raw data file of the current scanning layer according to the preset data slicing rule may include the following process.

The target file group corresponding to the raw data file of the current scanning layer may be determined based on a scanning layer identifier of the current scanning layer according to a first rule, wherein the first rule may include a correspondence between scanning layer identifiers and file groups.

In one embodiment, determining the target file group corresponding to the raw data file of the current scanning layer according to the preset data slicing rule may include the following process.

Whether a data capacity of a storage space of a current file group is greater than or equal to a preset data capacity threshold may be evaluated according to the third rule, wherein the third rule may include a data capacity threshold corresponding to a storage space of each file group.

If the data capacity of the storage space of the current file group is less than the data capacity threshold, the current file group may be determined as the target file group.

If the data capacity of the storage space of the current file group is greater than or equal to the data capacity threshold, a next file group may be determined as the target file group.

In one embodiment, determining the target file group corresponding to the raw data file of the current scanning layer according to the preset data slicing rule may include:

Whether a data capacity of a storage space of a current file group is greater than or equal to a preset data capacity threshold may be evaluated according to the third rule, wherein the third rule may include a data capacity threshold corresponding to a storage space of each file group.

If the data capacity of the storage space of the current file group is less than the data capacity threshold, the current file group may be determined as the target file group.

If the data capacity of the storage space of the current file group is greater than or equal to the data capacity threshold, a next file group may be determined as the target file group.

In one embodiment, the process for magnetic resonance scanning may further include following operations.

Whether a scanning completion command or a protocol sequence suspending command is received may be determined.

If the scanning completion command or the protocol sequence suspending command is received, a storage task may be terminated.

If neither the scanning completion command nor the protocol sequence suspending command is received, a raw data file of a next scanning layer may be obtained as the raw data file of the current scanning layer, and the target file group corresponding to the raw data file of the current scanning layer may be determined according to the preset data slicing rule.

In one embodiment, the process for magnetic resonance scanning may further include following operations.

In response to receiving the protocol sequence suspending command, the magnetic resonance device may be controlled to stop scanning a target object, a layer count of scanning layers that have been scanned may be recorded, a task execution log file may be generated, and a storage task may be terminated.

In one embodiment, the process for magnetic resonance scanning may further include following operations.

In response to receiving a suspending protocol sequence activation command, the task execution log file may be searched for, the layer count of scanning layers that have been scanned may be retrieved, and layers that have not been scanned may be scanned.

More descriptions of the operations of the process for magnetic resonance scanning in the above embodiments may be found in the descriptions of the process for data storage in magnetic resonance scanning above, which are not repeated here.

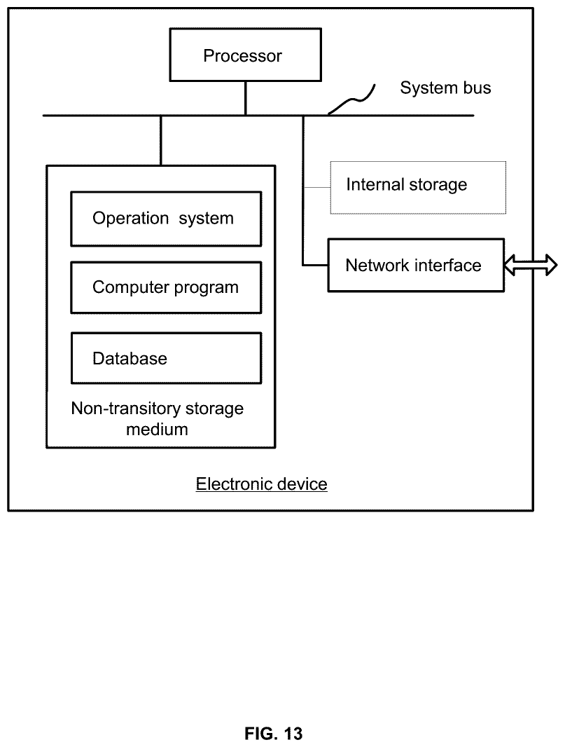

In one embodiment, an electronic device is provided, which may be a server. An exemplary internal structure of the electronic device is shown in FIG. 13. The electronic device may include at least one processor, a storage device, and a network interface connected via a system bus. The at least one processor of the electronic device may be configured to provide computing and control capabilities. The storage device of the electronic device may include a non-volatile storage medium and an internal storage. The non-volatile storage medium may be configured to store an operation system, one or more computer programs, and databases. The internal storage may provide an environment for running the operation system and one or more computer programs stored in the non-volatile storage medium. The database of the electronic device may be used to store raw data. The network interface of the electronic device may be configured to communicate with an external terminal via a network. When executed by the at least one processor, the one or more computer programs implement the method for data storage in magnetic resonance scanning or the method for magnetic resonance scanning.

It should be understood by those skilled in the art that the structure shown in FIG. 13 is only a schematic diagram of the relevant part of the structure associated with the present disclosure, and does not constitute a limitation on the computer device to which the present disclosure is applied. Specific computer devices may include more or fewer components than those shown in the drawing, or combine certain components, or have different arrangements of components.

In one embodiment, an electronic device is provided, which may include a storage device and at least one processor, wherein the storage stores one or more computer programs, and the at least one processor executes the one or more computer programs to implement the method for data storage in magnetic resonance scanning or the method for magnetic resonance scanning described in any of the embodiments above.

In one embodiment, a non-transitory computer-readable storage medium is provided, on which one or more computer programs may be stored. When executed by a processor, the one or more computer programs implement the method for data storage in magnetic resonance scanning or the method for magnetic resonance scanning described in any of the embodiments above.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure; For example, the terms “one embodiment,” “an embodiment,” and/or “some embodiments” mean that a particular feature, structure, or characteristic described in connection with the embodiment is included in at least one embodiment of the present disclosure; Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure;

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

As another example, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameter set forth in the written description and attached claims are approximations that may vary depending upon the desired properties sought to be obtained by a particular embodiment. In some embodiments, the numerical parameter should be construed in light of the number of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method implemented on at least one machine each of which has at least one processor and a storage device for data storage in magnetic resonance scanning, comprising:
obtaining a raw data file of a current scanning layer during scanning a target object using a magnetic resonance device;
determining a target file group corresponding to the raw data file of the current scanning layer according to a preset data slicing rule; and
storing data of the raw data file of the current scanning layer into a storage space corresponding to the target file group.

2. The method of claim 1, wherein the data slicing rule includes at least one of:
a first rule for grouping raw data files based on a layer count;
a second rule for grouping raw data files based on a scanning time; or
a third rule for grouping raw data files based on a data capacity.

3. The method of claim 2, wherein the determining a target file group corresponding to the raw data file of the current scanning layer according to a preset data slicing rule includes:
determining, according to the first rule, the target file group corresponding to the raw data file of the current scanning layer based on a scanning layer identifier of the current scanning layer, wherein the first rule includes a correspondence between scanning layer identifiers and file groups.

4. The method of claim 2, wherein the determining a target file group corresponding to the raw data file of the current scanning layer according to a preset data slicing rule includes:
determining, according to the second rule, whether a scanning time of the current scanning layer falls within a storage time interval corresponding to a current file group, wherein the second rule includes a storage time interval corresponding to each file group; and
if the scanning time of the current scanning layer falls within the storage time interval corresponding to the current file group, determining the current file group as the target file group; or if the scanning time of the current scanning layer exceeds the storage time interval corresponding to the current file group, determining a file group corresponding to a next storage time interval as the target file group.

5. The method of claim 2, wherein the determining a target file group corresponding to the raw data file of the current scanning layer according to a preset data slicing rule includes:
evaluating, according to the third rule, whether a data capacity of a storage space of a current file group is greater than or equal to a preset data capacity threshold, wherein the third rule includes a data capacity threshold corresponding to a storage space of each file group; and
if the data capacity of the storage space of the current file group is less than the data capacity threshold, determining the current file group as the target file group; or if the data capacity of the storage space of the current file group is greater than or equal to the data capacity threshold, determining a next file group as the target file group.

6. The method of claim 1, further comprising:
determining whether a scanning completion command or a protocol sequence suspending command is received; and
if the scanning completion command or the protocol sequence suspending command is received, terminating a storage task; or if neither the scanning completion command nor the protocol sequence suspending command is received, obtaining a raw data file of a next scanning layer as the raw data file of the current scanning layer, and determining the target file group corresponding to the raw data file of the current scanning layer according to the preset data slicing rule.

7. The method of claim 6, further comprising:
in response to receiving the protocol sequence suspending command,
controlling the magnetic resonance device to stop scanning the target object,
recording a layer count of scanning layers that have been scanned, and
generating a task execution log file.

8. The method of claim 7, further comprising:
in response to receiving a suspending protocol sequence activation command,
searching for the task execution log file,
retrieving the layer count of scanning layers that have been scanned, and
scanning layers that have not been scanned.

9. A method implemented on at least one machine each of which has at least one processor and a storage device for magnetic resonance scanning executed by a magnetic resonance device, comprising:
obtaining a target scanning protocol selected by a user via a protocol scanning interface;
navigating to a slice selection interface based on the target scanning protocol, obtaining a preset data slicing rule selected by the user via the slice selection interface, and scanning a target object to generate a raw data file of a current scanning layer; and
storing the raw data file of the current scanning layer according to the preset data slicing rule, and storing data of the raw data file of the current scanning layer into a storage space corresponding to a target file group.

10. The method of claim 9, further comprising:
detecting, during a scanning process, whether a protocol sequence suspending command is triggered;
in response to determining that the protocol sequence suspending command is triggered, switching a scanning sequence from a scanning state to a suspending state;
when the scanning sequence is in the suspending state, controlling the magnetic resonance device to stop scanning the target object and recording a completed scanning task and a raw data file corresponding to the completed scanning task, wherein the scanning task is associated with at least one of scanned layers of the magnetic resonance scanning and a magnetic resonance signal corresponding to each of the scanned layers;
detecting whether the user activates the scanning sequence; and
in response to determining that the user activates the scanning sequence,
determining an activation position of the scanning sequence based on a trigger state of the protocol sequence suspending command; and
continuing to perform subsequent scanning of the scanning sequence based on the activation position.

11. The method of claim 10, wherein the protocol sequence suspending command is a command pre-added to a scanning protocol.

12. The method of claim 10, wherein the completed scanning task and the raw data file corresponding to the completed scanning task are recorded in a task execution log file.

13. The method of claim 10, wherein the determining an activation position of the scanning sequence based on a trigger state of the protocol sequence suspending command includes:
in response to determining that the trigger state is a first trigger state, determining a first suspension position of a first scanning task, wherein the first scanning task is a currently executing scanning task recorded when the scanning sequence is suspended; and
determining the first suspension position as the activation position; or
in response to determining that the trigger state is a second trigger state, determining a second suspension position of a second scanning task, wherein the second scanning task is a preceding completed scanning task recorded when the scanning sequence is suspended; and
determining the second suspension position as the activation position.

14-15. (canceled)

16. A system for magnetic resonance scanning, comprising a protocol manager module, a checklist module, and a data storage module, wherein:
the protocol manager module is configured to obtain a target scanning protocol selected by a user via a protocol scanning interface and transmit the target scanning protocol to the checklist module;
the checklist module is configured to navigate to a slice selection interface based on the target scanning protocol, obtain a preset data slicing rule selected by the user via the slice selection interface, and scan a target object to generate a raw data file of a current scanning layer; and
the data storage module is configured to store the raw data file of the current scanning layer according to the preset data slicing rule.

17. The system of claim 16, further comprising a suspending module and an activation module, wherein:
the suspending module is configured to obtain a protocol sequence suspending command based on the protocol scanning interface and when the protocol sequence suspending command is received, control a magnetic resonance device to stop scanning the target object, record a layer count of scanning layers that have been scanned, generate a task execution log file, and transmit the task execution log file to the data storage module for storage; and
the activation module is configured to obtain a suspending protocol sequence activation command based on the protocol scanning interface, search for the task execution log file in the data storage module, retrieve the layer count of scanning layers that have been scanned, and scan layers that have not been scanned.

18. The system of claim 17, wherein:
the suspending module is further configured to:
detect, during a scanning process, whether a protocol sequence suspending command is triggered;
in response to determining that the protocol sequence suspending command is triggered, switch a scanning sequence from a scanning state to a suspending state;
when the scanning sequence is in the suspending state, control the magnetic resonance device to stop scanning the target object and record a completed scanning task and a raw data file corresponding to the completed scanning task, wherein the scanning task is associated with at least one of scanned layers of the magnetic resonance scanning, and a magnetic resonance signal corresponding to each of the scanned layers; and
the activation module is further configured to:
detect whether the user activates the scanning sequence; and
in response determining that the user activates the scanning sequence,
determine an activation position of the scanning sequence based on a trigger state of the protocol sequence suspending command; and
continue to perform subsequent scanning of the scanning sequence based on the activation position.

19. The system of claim 18, further comprising a protocol management module, and the protocol management module is configured to pre-add the protocol sequence suspending command in a scanning protocol.

20. The system of claim 18, wherein the suspending module is further configured to record the completed scanning task and the raw data file corresponding to the completed scanning task in the task execution log file.

21. The system of claim 18, wherein the activation module is further configured to:
in response to determining that the trigger state is a first trigger state, determine a first suspension position of a first scanning task, wherein the first scanning task is a currently executing scanning task recorded when the scanning sequence is suspended; and
determine the first suspension position as the activation position; or
in response to determining that the trigger state is a second trigger state, determine a second suspension position of a second scanning task, wherein the second scanning task is a preceding completed scanning task recorded when the scanning sequence is suspended; and
determine the second suspension position as the activation position.

22. (canceled)

23. The system of claim 18, wherein the data storage module is configured to:
obtain a data slicing rule configured by the user, wherein the data slicing rule includes at least: a first rule for grouping raw data files based on a layer count, a second rule for grouping raw data files based on a scanning time, or a third rule for grouping raw data files based on a data volume; and
split the raw data files according to the data slicing rule and store the split raw data files in a storage space.

24-25. (canceled)
